(19) United States
(12) Patent Application Publication
(10) Pub. No.: US 2023/0145523 A1
(43) Pub. Date: May 11, 2023

(54) MEDICAL IMAGE PROCESSING APPARATUS, X-RAY CT APPARATUS, MEDICAL IMAGE PROCESSING METHOD AND NON-VOLATILE STORAGE MEDIUM STORING PROGRAM

(71) Applicant: CANON MEDICAL SYSTEMS CORPORATION, Otawara-shi (JP)

(72) Inventors: Masakazu MATSUURA, Nasushiobara (JP); Jian ZHOU, Vernon Hills, IL (US); Zhou YU, Vernon Hills, IL (US)

(73) Assignee: CANON MEDICAL SYSTEMS CORPORATION, Otawara-shi (JP)

(21) Appl. No.: 17/680,048

(22) Filed: Feb. 24, 2022

Related U.S. Application Data
(60) Provisional application No. 63/277,751, filed on Nov. 10, 2021.

(51) Int. Cl. G06T 11/00; G06T 7/30; A61B 6/00; A61B 6/03

(52) U.S. Cl. CPC G06T 11/008; G06T 7/30; A61B 6/5258; A61B 6/032; G06T 2207/10081; G06T 2207/20081; G06T 2207/10116; A61B 6/563

(57) ABSTRACT

A medical image processing apparatus according to an embodiment includes processing circuitry configured to acquire a radiographic image of a subject and acquire a post-processing image with reduced steak artifacts by applying a model that reduces streak artifacts to the radiographic image, wherein the model is generated by machine learning that uses, as a training data pair, a first radiographic image and a second radiographic image based on artifact generation processing for generating streak artifacts.

### FIELD

Embodiments described herein relate generally to a medical image processing apparatus, an X-ray CT apparatus, a medical image processing method and a non-volatile storage storing a program.

### BACKGROUND

Various radiographic images that are acquired by a radiographic diagnosis apparatus, such as a computed tomography (CT) apparatus, a positron emission computed tomography (PET) apparatus, a single photon emission computed tomography (SPECT) apparatus, or a chest X-ray or X-ray angiography apparatus, are known.

Such a radiographic image may contain streak artifacts due to various factors. For example, in a process of generating a radiographic image or image processing after the generation, high-frequency components are sometimes enhanced for the purpose of increasing a spatial resolution. In such processing, aliasing sometimes occurs and this sometimes appears as streak artifacts in a radiographic image. When generating a radiographic image by reconstruction processing, streak artifacts sometimes appear in the radiographic image due to mechanical accuracy in an imaging unit of a radiographic diagnosis apparatus.

### BRIEF DESCRIPTION OF THE DRAWINGS

FIG. 1A is a block diagram illustrating an example of a configuration of a medical image processing system according to a first embodiment;

FIG. 1B is a block diagram illustrating an example of a configuration of a medical image processing apparatus according to the first embodiment;

FIG. 2 is a block diagram illustrating an example of a configuration of an X-ray CT apparatus according to the first embodiment;

FIG. 3 is a diagram illustrating a process flow according to the first embodiment;

FIG. 4 is a diagram illustrating streak artifacts according to the first embodiment; and

FIG. 5 is a block diagram illustrating an example of a configuration of an X-ray CT apparatus according to a seventh embodiment.

### DETAILED DESCRIPTION

With reference to the accompanying drawings, an embodiment of a medical image processing apparatus, an X-ray CT apparatus, a medical image processing method and a non-volatile storage storing a program will be described below.

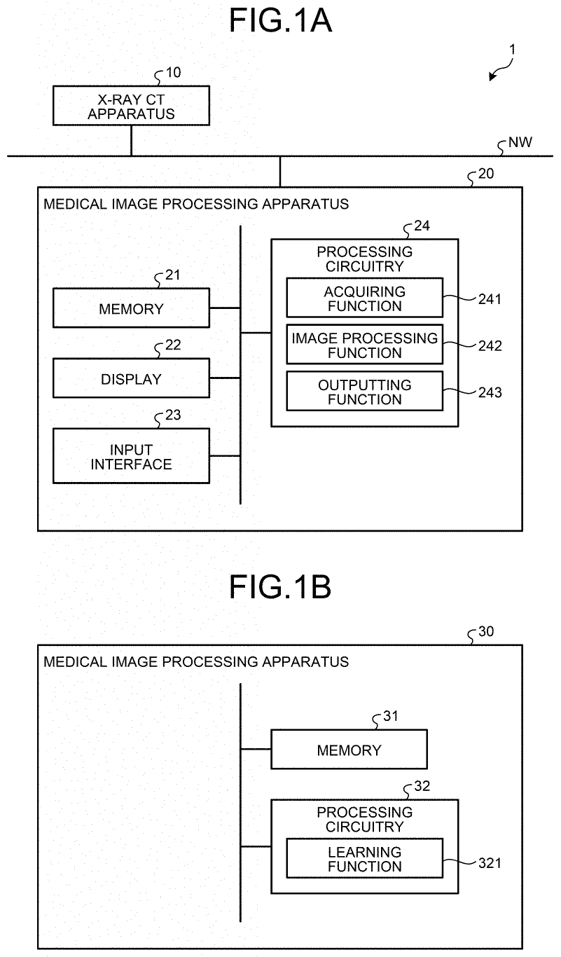

In the present embodiment, a medical image processing system 1 illustrated in FIG. 1A and a medical image processing apparatus 30 illustrated in FIG. 1B will be described as an example. A medical image processing apparatus 20 contained in the medical image processing system 1 reduces streak artifacts in a radiographic image using a model M1 that is generated by the medical image processing apparatus 30. FIG. 1A is a block diagram illustrating an example of a configuration of the 1 according to a first embodiment. FIG. 1B is a block diagram illustrating an example of a configuration of a medical image processing apparatus according to the first embodiment.

As illustrated in FIG. 1A, the 1 includes an X-ray CT apparatus 10 and the medical image processing apparatus 20. In the embodiment, the X-ray CT apparatus 10 will be described as an example of a radiographic diagnosis apparatus. In other words, in the embodiment, an X-ray CT image will be described as an example of a radiographic image. The X-ray CT apparatus 10 and the medical image processing apparatus 20 are connected to each other via a network NW.

As long as connection is enabled via the network NW, the X-ray CT apparatus 10 and the medical image processing apparatus 20 are set any locations. For example, the X-ray CT apparatus 10 and the medical image processing apparatus 20 may be set in different facilities. In other words, the NW may consists of a local area network closed in the facility or may be a network via the Internet. Communication between the X-ray CT apparatus 10 and the medical image processing apparatus 20 may be performed via another apparatus, such as an image storage device, or may be performed directly not via any other device. For example, a server of PACS (Picture Archiving and Communication System) is exemplified as an example of such an image storage device.

For example, as illustrated in FIG. 1A, the medical image processing apparatus 20 includes a memory 21, a display 22, an input interface 23 and processing circuitry 24.

The memory 21 is implemented by, for example, a semiconductor memory, such as a random access memory (RAM) or a flash memory, a hard disk or an optical disk. For example, the memory 21 stores a program for circuitry contained in the medical image processing apparatus 20 to implement its function. The memory 21 stores X-ray CT images that are acquired by the X-ray CT apparatus 10. The memory 21 stores the model M1 that reduces streak artifacts. The model M1 will be described below. The memory 21 may be implemented by a server group (cloud) that is connected to the medical image processing apparatus 20 via the network NW.

The display 22 displays various types of information. For example, the display 22 displays an X-ray CT image whose streak artifacts are reduced by the processing circuitry 24. For example, the display 22 is a liquid crystal display or a cathode ray tube (Cathode Ray Tube) display. The display 22 may be a tablet terminal enabling radio communication with the body of the medical image processing apparatus 20.

The input interface 23 receives various input operations from the user, converts the received input operation into an electric signal and outputs the electric signal to the processing circuitry 24. For example, the input interface 23 is implemented by a mouse and a keyboard, a trackball, a switch, a button, a joystick, a touch pad via which an input operation is made by a contact on an operation screen, a touch screen on which a screen image and a touch pad are integrated, a non-contact input circuit using an optical sensor, or an audio input circuit. The input interface 23 may consist of a tablet terminal enabling radio communication with the medical image processing apparatus 20. The input interface 23 may be a circuit that receives an input operation from the user by motion capture. For example, the input interface 23 is able to receive the body motion or lines of sight of the user as an input operation by processing signals that are acquired by a tracker and/or images that are acquired from the user. The input interface 23 is not limited to one including physical operational parts, such as a mouse or a keyboard. For example, examples of the input interface 23 includes an electric signal processing circuit that receives an electric signal corresponding to an input operation from an external input device provided independently of the medical image processing apparatus 20 and outputting the electric signal to the processing circuitry.

The processing circuitry 24 controls entire operations of the medical image processing apparatus 20 by executing an acquiring function 241, an image processing function 242, and an outputting function 243. The acquiring function 241 is an example of an acquisition unit. The image processing function 242 is an example of an image processor.

For example, the processing circuitry 24 reads a program corresponding to the acquiring function 241 from the memory 21 and executes the program, thereby acquiring an X-ray CT image. For example, the acquiring function 241 acquires, via the network NW, X-ray CT images that are acquired by the X-ray CT apparatus 10. Alternatively, the acquiring function 241 may, via the network NW, acquire projection data that is acquired by the X-ray CT apparatus 10. In this case, the acquiring function 241 is able to reconstruct an X-ray CT image using the acquired projection data.

The processing circuitry 24 reads a program corresponding to the image processing function 242 from the memory 21 and executes the program, thereby applying the model M1 to a radiographic image and acquires a post-processing image in which streak artifacts are reduced. The processing circuitry 24 reads a program corresponding to the outputting function 243 from the memory 21 and executes the program, thereby displaying the post-processing image in which streak artifacts on the display 22. Details of each function of the processing circuitry 24 will be described below.

In the medical image processing apparatus 20 illustrated in FIG. 20, each of the processing functions is stored in a form of a computer-executable program in the memory 21. The processing circuitry 24 is a processor that reads a program from the memory 21 and executing the program, thereby implementing a function corresponding to each program. In other words, the processing circuitry 24 having read a program includes the function corresponding to the read program.

FIG. 1A illustrates that the single processing circuitry 24 implements the acquiring function 241, the image processing function 242 and the outputting function 243; however, a plurality of independent processors may be combined to configure the processing circuitry 24 and the respective processors may execute the programs, thereby implementing the functions. Each of the processing functions that the processing circuitry 24 includes may be distributed or integrated to or into a single or a plurality of processing circuits as appropriate and be implemented.

The processing circuitry 24 may implement the functions using a processor of an external apparatus that is connected via the network NW. For example, the processing circuitry 24 reads the program corresponding to each of the functions from the memory 21 and executes the program and uses a server group (cloud) that is connected to the medical image processing apparatus 20 via the network NW as a calculation resource, thereby implementing each of the functions illustrated in FIG. 1A.

The medical image processing apparatus 30 will be described next. As illustrated in FIG. 1B, the medical image processing apparatus 30 includes a memory 31 and processing circuitry 32.

The memory 31 can be configured similarly to the memory 21 described above. For example, the memory 31 saves training data to be described below and circuitry contained in the medical image processing apparatus 30 stores a program for implementing a function thereof.

The processing circuitry 32 reads a program corresponding to a learning function 321 from a memory 141 and executes the program, thereby generating the model M1. The learning function 321 is an example of a learning unit. Details of the learning function 321 will be described below.

In the medical image processing apparatus 30 illustrated in FIG. 1B, each processing function is stored in a form of a computer-executable program in the memory 31. The processing circuitry 32 is a processor that reads a program from the memory 31 and executes the program, thereby implementing a function corresponding to each program. In other words, the processing circuitry 32 having read a program includes the function corresponding to the read program.

FIG. 1B illustrates that the single processing circuitry 32 implements the learning function 321; however, a plurality of independent processors may be combined to configure the processing circuitry 32 and the respective processors may execute programs, thereby implementing the function. Processing functions that the processing circuitry 32 includes may be distributed or integrated to or into a single or a plurality of processing circuits and be implemented.

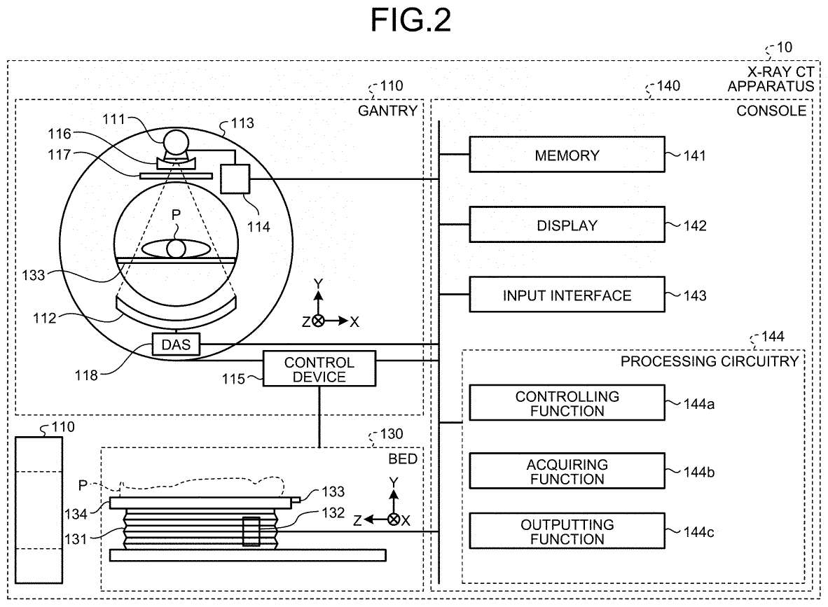

The X-ray CT apparatus 10 will be described next using FIG. 2. FIG. 2 is a block diagram illustrating an example of the X-ray CT apparatus 10 according to the first embodiment. For example, the X-ray CT apparatus 10 includes a gantry 110, a bed 130, and a console 140.

In FIG. 2, a rotational axis of a rotation frame 113 in a non-tilt state or a longitudinal direction of a couch top 133 of the bed 130 is Z-axis direction. An axial direction orthogonal to the Z-axis direction and that is parallel to a floor surface is an X-axis direction. An axial direction that is orthogonal to the Z-axis direction and that is perpendicular to the floor surface is a Y-axis direction. FIG. 2 is a drawing of the gantry 110 from multiple directions for description and illustrates the case where the X-ray CT apparatus 10 includes the single gantry 110.

The gantry 110 includes an X-ray tube 111, an X-ray detector 112, the rotation frame 113, an X-ray high-voltage device 114, a control device 115, a wedge 116, a collimator 117, and a data acquisition system (DAS) 118.

The X-ray tube 111 is a vacuum tube including a cathode (filament) that generates thermoelectrons and an anode (target) that generates X-rays in response to collision of the thermoelectrons. The X-ray tube 111 applies thermoelectrons from the cathode to the anode in response to application of a high voltage from the X-ray high voltage device 114, thereby generating X-rays to be applied to the subject P.

The X-ray detector 112 detects X-rays having been applied from the X-ray tube 111 and having passed through the subject P and outputs a signal corresponding to the detected x-ray dosage to the DAS 118. The X-ray detector 112, for example, includes a plurality of detection element rows in which a plurality of detection elements are arrayed in a channel direction (channel direction) along an arc about a focal point of the X-ray tube 111. The X-ray detector 112, for example, has a configuration in which a plurality of detection element rows in which a plurality of detection elements are arrayed in the channel direction are arrayed in the row direction (slice direction or row direction).

For example, the X-ray detector 112 is an indirect transformation detector including a grid, a scintillator array, and an optical sensor array. The scintillator array includes a plurality of scintillators. The scintillator includes a scintillator crystal that outputs light of a photon quantity corresponding to a dosage of incident X-rays. The grid is arranged on a surface of the scintillator array on the side of incidence of X-rays and includes an X-ray shield that absorbs scattering X-rays. The grid is sometimes referred to as a collimator (one-dimensional collimator or a two-dimensional collimator). An optical sensor array has a function of transformation into an electric signal corresponding to the amount of light from the scintillator and includes an optical sensor, such as a photodiode. The X-ray detector 112 may be a direct transformation detector including a semiconductor device that transforms incident X-rays into an electric signal.

The rotation frame 113 is an annular frame that supports the X-ray tube 111 and the X-ray detector 112 such that the X-ray tube 111 and the X-ray detector 112 are opposed to each other and that is caused by the control device 115 to rotate the X-ray tube 111 and the X-ray detector 112. For example, the rotation frame 113 is a cast made of aluminum. The rotation frame 113 is also able to further support the X-ray high-voltage device 114, the wedge 116, the collimator 117, the DAS 118, etc., in addition to the X-ray tube 111 and the X-ray detector 112. Furthermore, the rotation frame 113 is capable of further supporting various structures not illustrated in FIG. 2. The rotation frame 113 and parts that rotate together with the rotation frame 113 are also referred to as a rotation unit.

The X-ray high-voltage device 114 includes electric circuits, such as a transformer and a rectifier, and includes a high-voltage generation device that generates a high voltage to be applied to the X-ray tube 111 and an X-ray control device that performs control on an output voltage corresponding to the X-rays that are generated by the X-ray tube 111. The high-voltage generation device may be a transformer type or an inverter type. The X-ray high-voltage device 114 may be provided in the rotation frame 113 or 114 or may be provided in a fixed frame not illustrated in the drawing.

The control device 115 includes processing circuitry including a central processing unit (CPU), etc., and a drive mechanism, such as a motor, an actuator, etc. The control device 115 receives an input signal from an input interface 143 and controls operations of the gantry 110 and the bed 130. For example, the control device 115 performs control on rotation of the rotation frame 113, the tilt of the gantry 110, and operations of the bed 130. For example, the control device 115 causes the rotation frame 113 to rotate about an axis parallel to the X-axis direction according to inclination angle (tilt angle) information as the control of tilting the gantry 110. The control device 115 may be provided in the gantry 110 or may be provided in the console 140.

The wedge 116 is an X-ray filter for adjusting the dosage of X-rays applied from the X-ray tube 111. Specifically, the wedge 116 is an X-ray filter that attenuates X-rays that are applied from the X-ray tube 111 such that the X-rays applied to the subject P from the X-ray tube 111 have a predetermined distribution. For example, the wedge 116 is a wedge filter or a bow-tie filter and is manufactured by processing aluminum into a given target angle and a given thickness.

The collimator 117 includes a lead plate for narrowing the area of radiation with X-rays having been transmitted through the wedge 116 and forms a slit according to combination of a plurality of lead plates, or the like. The collimator 117 is sometimes referred to as an X-ray diaphragm. FIG. 2 illustrates the case where the wedge 116 is arranged between the X-ray tube 111 and the collimator 117; however, the collimator 117 may be arranged between the X-ray tube 111 and the wedge 116. In this case, the wedge 116 transmits X-rays that are applied from the X-ray tube 111 and whose area of radiation is restricted by the collimator 117 and attenuates the X-rays.

The DAS 118 acquires signals of X-rays that are detected by the respective detection elements that the X-ray detector 112 includes. For example, the DAS 118 includes an amplifier that performs amplification processing on the electric signal that is output from each of the detection elements and an A/D converter that converts the electric signal into a digital signal and generates detection data. The DAS 118 is implemented by, for example, a processor.

The data that is generated by the DAS 118 is transmitted from a transmitter with a light emitting diode (LED) that is provided in the rotation frame 113 by optical communication to a receiver with a photodiode that is provided in a non-rotation part of the gantry 110 (for example, the fixed frame, or the like, of which illustration in FIG. 2 is omitted) and is transferred to the console 140. The non-rotation part is, for example, the fixed frame that supports the rotation frame 113 rotatably. A method of transmitting data from the rotation frame 113 to the gantry 110 is not limited to optical communication, and any non-contact data transmission system may be employed or contact data transmission system may be employed.

The bed 130 is a device on which the subject P to be scanned is laid and that moves the subject P and includes a base 131, a couch drive device 132, the couch top 133, and a support frame 134. The base 131 is a casing that supports the support frame 134 movably in the vertical direction. The couch drive device 132 is a drive mechanism that moves the couch top 133 on which the subject P is laid in a longitudinal direction of the couch top 133 and includes a motor and an actuator. The couch top 133 that is provided on the top surface of the support frame 134 is a board on which the subject P is laid. The couch drive device 132 may move, in addition to the couch top 133, the support frame 134 may be moved in the longitudinal direction of the couch top 133.

The console 140 includes the memory 141, a display 142, the input interface 143, and processing circuitry 144. The console 140 is described independently from the gantry 110; however, the console 140 or part of each component of the console 140 may be contained in the gantry 110.

The memory 141 can be configured similarly as the memory 21. For example, the memory 141 saves various types of data acquired from the subject P and stores programs for the circuitry contained in the X-ray CT apparatus 10 to implement the functions of the circuitry.

The display 142 can be configured similarly to the display 22 described above. For example, the display 142 is capable of displaying a GUI for receiving various instructions, settings, etc., from the user.

The input interface 143 can be configured similarly as the input interface 23 described above. For example, the input interface 143 receives various input operations from the user, transforms the received input operations into electric signals, and outputs the electric signals to the processing circuitry 144.

The processing circuitry 144 executes a controlling function 144a, an acquiring function 144b, and an outputting function 144c, thereby controlling entire operations of the X-ray CT apparatus 10.

For example, the processing circuitry 144 reads a program corresponding to the controlling function 144a from the memory 141 and executes the program, thereby controlling various function, such as the acquiring function 144b and the outputting function 144c, based on various input operations that are received from the user via the input interface 143.

For example, the processing circuitry 144 reads a program corresponding to the acquiring function 144b from the memory 141 and executes the program, thereby executing scanning on the subject P. For example, the acquiring function 144b controls the X-ray high-voltage device 114, thereby supplying a high-voltage to the X-ray tube 111. Thus, the X-ray tube 111 generates X-rays to be applied to the subject P. The acquiring function 144b controls the couch drive device 132, thereby moving the subject P into an imaging port of the gantry 110. The acquiring function 144b adjusts the position of the wedge 116 and the degree of opening and position of the collimator 117, thereby controlling the distribution of X-rays to be applied to the subject P. The acquiring function 144b controls the control device 115, thereby rotating the rotation unit. While the acquiring function 144b is executing scanning, the DAS 118 acquires signals of X-rays from the respective detection elements in the X-ray detector 112 and generates detection data.

The acquiring function 144b performs pre-processing on the detection data that is output from the DAS 118. For example, the acquiring function 144b performs pre-processing, such as logarithmic transformation and offset correction processing, sensitivity correction processing between channels and beam hardening correction processing, on the detection data that is output from the DAS 118. The data on which pre-processing has been performed is also referred to as raw data. The detection data before performing of the pre-processing and the raw data after performing of the pre-processing are collectively referred to also as projection data.

For example, the processing circuitry 144 reads a program corresponding to the outputting function 144c and executes the program, thereby outputting various types of data acquired from the subject P. For example, the outputting function 144c transmits data that is acquired by executing scanning on the subject P to the medical image processing apparatus 20 via the network NW. For example, the outputting function 144c performs control on display by the display 142.

In the X-ray CT apparatus 10 illustrated in FIG. 2, each of the processing functions is stored in a form of a computer-executable program in the memory 141. The processing circuitry 144 is a processor that reads a program from the memory 141 and executes the program, thereby implementing a function corresponding to each program. In other words, the processing circuitry 144 having read the program includes the function corresponding to the read program.

FIG. 2 illustrates that the single processing circuitry 144 implements the controlling function 144a, the acquiring function 144b and the outputting function 144c; however, a plurality of independent processors may be combined to configure the processing circuitry 144 and the respective processors may execute the programs, thereby implementing the functions. Each of the processing functions that the processing circuitry 144 includes may be distributed or integrated to or into a single or a plurality of processing circuits as appropriate and be implemented.

The processing circuitry 144 may implement the functions using a processor of an external device that is connected via the network NW. For example, the processing circuitry 144 reads the program corresponding to each of the functions from the memory 141 and executes the program and uses a server group (cloud) that is connected to the X-ray CT apparatus 10 via the network NW as a calculation resource, thereby implementing each of the functions illustrated in FIG. 2.

Streak artifacts occurring in an X-ray CT image will be described next. There are some factors of generation of streak artifacts and processing of enhancing high-frequency components can be taken as an example. For example, performing reconstruction processing in which high-frequency components are enhanced at the time of generation of an X-ray CT image sometimes causes streak artifacts. For example, according to a reconstruction protocol on bones, or the like, emphasizing a spatial resolution, a reconstruction function that enhances high-frequency components is sometimes used. When an X-ray CT image is reconstructed by MBIR (Model-Based Interactive Reconstruction), a parameter that more emphasizes the spatial resolution than noise is sometimes set. After generation of an X-ray image, image processing (post processing) for increasing the spatial resolution is sometimes performed. Excessively enhancing high-frequency components sometimes causes aliasing, which sometimes appears as streak artifacts in a radiographic image.

Mechanical accuracy in an imaging unit of a radiographic diagnosis apparatus is taken as another factor of generation of streak artifacts. The imaging unit of the radiographic diagnosis apparatus is a part of the radiographic diagnosis apparatus that is driven for imaging and, in the case of the X-ray CT apparatus 10, the gantry 110 containing the X-ray tube 111 and the X-ray detector 112 corresponds. The imaging unit of the radiographic diagnosis apparatus is also referred to as a radiographic imaging system.

Specifically, in reconstruction of an X-ray CT image, calculation is performed in each time point during imaging, assuming that the X-ray tube 111 and the X-ray detector 112 are in given positons. When the actual positions of the X-ray tube 111 and the X-ray detector 112 are shifted from the given positions, calculation is performed with the positons of the X-ray tube 111 and the X-ray detector 112 being incorrect and streak artifacts sometimes appear in a radiographic image.

Manufacturing the X-ray CT apparatus 10 is performed such that such a positional shift of the imaging unit does not occur and, when the X-ray CT apparatus 10 is conveyed to a hospital or is installed, a positional shift of the imaging unit sometimes occurs. In order to deal with the positional shift of the imaging unit, alignment is sometimes performed on the X-ray tube 111 and the X-ray detector 112. Specifically, when the X-ray CT apparatus 10 is installed or in regular maintenance, adjustment on the positions of the X-ray tube 111 and the X-ray detector 112 is sometimes performed. Such alignment is however performed by a service staff manually and a shift that is too small to adjust manually tends to remain. Furthermore, because of repeated use of the X-ray CT apparatus 10, positional shifts of the imaging unit sometimes occur over time or increase.

Correcting data such that the positional shift of the imaging unit does not have an effect on an X-ray CT image is also considered. For example, when an X-ray CT image is reconstructed, processing of forward projection and back projection based on acquired projection data is performed. A technique of estimating and correcting a positional shift of the imaging unit during an operation of the forward projection and back projection is known; however, it is not easy to estimate a positional shift of the imaging unit and it is often not possible to correct the positional shift sufficiently.

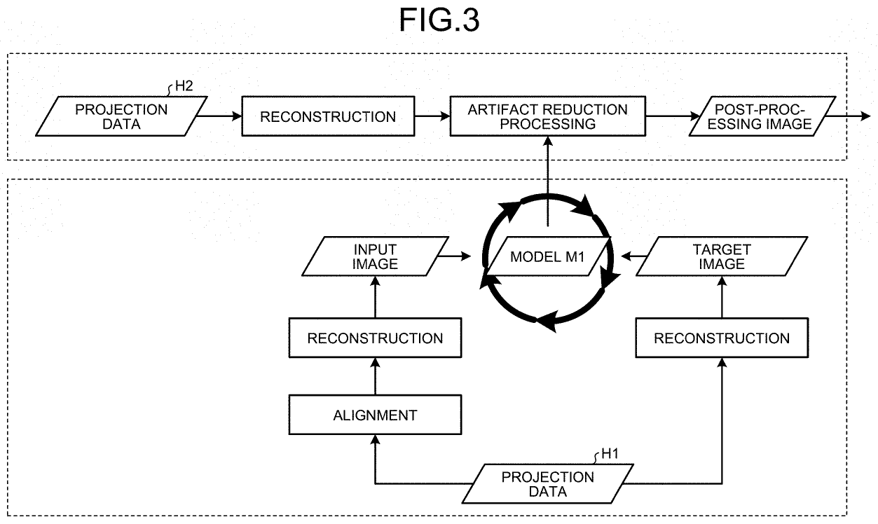

The image processing function 242 in the medical image processing apparatus 20 thus applies the model M1 to an X-ray CT image, thereby acquiring a post-processing image with reduced streak artifacts. Generation of the model M1 and application of the model M1 to an X-ray CT image will be described below using FIG. 3. FIG. 3 is a diagram illustrating a process flow according to the first embodiment.

A lower view in FIG. 3 illustrates a learning phase. Specifically, the lower view in FIG. 3 illustrates a process of generating the model M1 that reduces streak artifacts that is performed by the learning function 321. For example, the learning function 321 first acquires projection data H1. The projection data H1 may be data that is acquired by the X-ray CT apparatus 10 or data that is acquired by another X-ray CT apparatus. The projection data H1 may be acquired on a phantom imitating a human body.

For example, the learning function 321 is capable of acquiring the projection data H1 via the network NW. The learning function 321 is also able to acquire the projection data H1 via, for example, any storage medium without connecting to the network NW.

The learning function 321 reconstructs the projection data H1 and generates a target image. A method of reconstruction is not particularly limited. For example, the learning function 321 is capable of generating a target image by performing reconstruction processing using a filter correction back projection method, and a successive approximation reconstruction method. The learning function 321 is able to generate a target image by performing reconstruction processing by AI (Artificial Intelligence). For example, the learning function 321 generates an X-ray CT image by a DLR (Deep learning Reconstruction) method. A target image is an example of a first radiographic image.

The learning function 321 performs alignment on the projection data H1, reconstructs the projection data H1 after alignment, and generates an input image. As in the case of a target image, a reconstruction method is not particularly limited. The input image is an example of a second radiographic image.

Alignment is an example of an artifact generation processing for generating streak artifacts and is a process of applying components corresponding to a positional shift of the imaging unit to projection data. For example, the projection data H1 is considered as data containing three-dimensional information in the channel direction, the row direction, and a direction of application of X-rays from the X-ray tube 111 (view direction) in the X-ray detector 112. The learning function 321 applies components corresponding to a positional shift in at least one of the channel direction, the row direction and the view direction to the projection data H1.

Hereinafter, “c” is put as a coordinate of the channel direction, “r” is put as a coordinate of the row direction, and “v” is put as a coordinate of the view direction. In the projection data H1, the signal that is detected by each detection element of the X-ray detector 112 can be associated with a set of coordinates (c,r,v). Hereinafter, a signal s1 is put as a signal that is detected by a detection element d1 in the X-ray detector 112 and the signal s1 is described as one that is associated with a set of coordinates (c1,r1,v1). In this case, by associating the signal s1 with the setoff coordinates (c2,r1,v1), the learning function 321 is able to apply components corresponding to a positional shift in the channel direction to the projection data H1. More specifically, by assuming that the center of each detection element of the X-ray detector 112 is shifted in the channel direction by, for example, few mm, the learning function 321 is able to apply components corresponding to the positional shift in the channel direction to the projection data H1. The case where the components corresponding to the positional shift in the channel direction are applied to the projection data H1 has been described; however, components corresponding to the positional shifts in the row direction and the view direction instead of or in addition to the channel direction may be applied.

Reconstruction of the projection data H1 after alignment is performed and accordingly streak artifacts are generated in the input image. On the other hand, no streak artifact is generated in the target image or the target image has more reduced streak artifacts than in the input image.

As illustrated in FIG. 3, the learning function 321 generates the model M1 by machine learning that uses the input image and the training image as a training data pair. For example, the model M1 consists of a deep convolution neural network (DCNN). In other words, the learning function 321 causes the DCNN to learn using the input image and the target image as the training data pair, thereby generating the model M1.

For example, the learning function 321 generates the model M1 by adjusting parameters such that the DCNN that receives an input of the input image is able to output a preferable result. For example, an output image with reduced streak artifacts of the input image is output from an output layer of the DCNN having received the input of the input image. The learning function 321 adjusts the parameters of the DCNN to minimize a function (error function) representing closeness between the output image and the target image. For example, learning of the DCNN is executed off-line.

The model M1 has been described as the DCNN; however, another type of machine learning engine may be employed. For example, the model M1 may be a neural network, such as a whole binding neural network or a recurrent neural network (RNN).

It is preferable that the same reconstruction method and conditions be used between the input image reconstruction processing and the target image reconstruction processing. This enables elements (such as the noise level) other than streak artifacts to be approximately equal between the input image and the target image and consequently enables the model M1 to learn efficiently.

The model M1 that is generated by the learning function 321 is transmitted to the medical image processing apparatus 20 via the network NW, any storage medium, or the like, and is stored in the memory 21. The image processing function 242 is capable of reading the model M1 from the memory 21 and executing processing of reducing streak artifacts.

The upper view in FIG. 3 illustrates a deduction phase. Specifically, the upper view in FIG. 3 illustrates a series of sets of processing until acquisition of a post-processing image with streak artifacts reduced from the X-ray CT image of the subject P and provision of the image to the user, such as a doctor.

First of all, the acquiring function 241 acquires an X-ray CT image of the subject P. Specifically, first of all, the X-ray CT apparatus 10 capture images of the subject P and acquires projection data H2. The acquiring function 241 acquires the projection data H2 via the network NW, performs reconstruction processing, and generates an X-ray CT image of the subject P. As in the case of the target image and the input image described above, a method of reconstruction is not particularly limited. The case where the acquiring function 241 executes the reconstruction processing has been described; however, the acquiring function 241 may acquire an X-ray CT image that is reconstructed by another apparatus, such as the X-ray CT apparatus 10, via the network NW.

The image processing function 242 then applies the model M1 to the X-ray CT image, thereby acquiring a post-processing image with reduced streak artifacts. The outputting function 243 outputs the post-processing image. For example, the outputting function 243 causes the post-processing image to be displayed on the display 22. For example, the outputting function 243 transmits the post-processing image to another device via the network NW. In this case, the post-processing image is displayed on the another device and is provided to the user, such as a doctor. For example, the outputting function 243 may transmit the post-processing image to the X-ray CT image and the display 142 may display the post-processing image.

As described above, in the first embodiment, the acquiring function 241 acquires an X-ray CT image of the subject P. The image processing function 242 applies the model M1 that reduces streak artifacts to the X-ray CT image, thereby acquiring a post-processing image with reduced streak artifacts. The model M1 is generated by machine learning that uses a target image and an input image based on the artifact generation processing for generating streak artifacts as a training data pair. This enables the medical image processing apparatus 20 to reduce streak artifacts in the X-ray CT image.

The medical image processing apparatus 20 is able to increase the spatial resolution in the X-ray CT image. In other words, enhancing high-frequency components excessively sometimes generates streak artifacts and thus the processing of generating an X-ray CT image and image processing after the generation are in general controlled such that high-frequency components are not enhanced too much. On the other hand, according to the medical image processing apparatus 20, because, even when streak artifacts are generated, it is possible to remove or reduce the streak artifacts, it is possible to sufficiently enhance the high-frequency components in the processing of generating an X-ray CT image or the image processing after the generation.

In the first embodiment, the case where the input image and the target image for the model M1 to learn are generated from the single projection data H1 has been described. In the second embodiment, the case where an input image and a target image are generated from different sets of projection data, respectively, will be described.

For example, the learning function 321 acquires a target image based on projection data H111 and acquires an input image based on projection data H112 that is different from the projection data H111. The projection data H111 is an example of first projection data. The projection data H112 is an example of second projection data. The projection data H111 and the projection data H112 are, for example, acquired by the X-ray CT apparatus 10 by capturing an image of the same phantom for a plurality of times.

In this case, the learning function 321 reconstructs the projection data H111 and generates a target image. The learning function 321 performs alignment on the projection data H112, reconstructs the projection data H112 after alignment, and generates an input image. The learning function 321 then generates a model M1 by machine learning that uses the input image and the target image as a training data pair. Thereafter, using the model M1, the image processing function 242 is able to perform processing of reducing streak artifacts on an X-ray CT image of the subject P.

For the first and second embodiments, the case where streak artifacts are generated in the input image by performing alignment after acquiring the projection data has been described. On the other hand, in the third embodiment, the case where acquiring projection data while performing alignment generates streak artifacts in an input image will be described.

In other words, in the first and second embodiments, the case where, assuming that there is a positional shift of the imaging unit, such as the X-ray tube 111 and the X-ray detector 112, components corresponding to the positional shift are virtually applied after projection data is acquired has been described. In the third embodiment, when projection data is acquired, a positional shift of the imaging unit, such as the X-ray tube 111 and the X-ray detector 112, is actually generated and thus components corresponding to the positional shift of the imaging unit is applied to the projection data.

For example, the learning function 321 acquires a target image based on projection data H121 and acquires an input image based on projection data H122 that is different from the projection data H121. The projection data H121 is an example of the first projection data. The projection data H122 is an example of the second projection data. The projection data H121 and the projection data H122 are acquired by the X-ray CT apparatus 10 by capturing an image of the same phantom for multiple times.

For example, after the projection data H121 is acquired, alignment on the imaging unit of the X-ray CT apparatus 10 is performed such that a positional shift is caused. For example, a practitioner, or the like, is able to shift the position in which the X-ray detector 112 is installed from a given position by approximately few mm in the channel direction. Accordingly, components corresponding to the positional shift in the channel direction are applied to the acquired projection data H122 to be acquired and streak artifacts are generated in an input image that is reconstructed based on the projection data H122. The learning function 321 generates the model M1 by machine learning that uses an input image and a target image as a training data pair. Thereafter, using the model M1, the image processing function 242 is able to execute processing of reducing streak artifacts on the X-ray CT image of the subject. The case where components corresponding to the positional shift in the channel direction are applied has been described; however, components corresponding to positional shifts in the row direction and the view direction instead of or in addition to the channel direction may be applied.

In the first and second embodiments, the case where alignment is performed after acquisition of the projection data, that is, before the reconstruction process and, in the third embodiment, the case where streak artifacts are generated in the input image by performing alignment at the time of acquisition of the projection data has been described. On the other hand, in a fourth embodiment, the case where streak artifacts are generated in an input image by performing alignment at the time of reconstruction will be described.

For example, the learning function 321 acquires a target image by reconstructing projection data H131 and acquires an input image by reconstructing the projection data while performing alignment on the projection data H131. For example, in the reconstruction method such as the filter correction back projection, calculations of forward projection and back projection are performed repeatedly. In forward projection and back projection, a tube position (focal point position in the X-ray tube 111) is set. The learning function 321 is able to generate streak artifacts in the input image that is reconstructed by performing forward projection and back projection with the tube position being shifted from a given position.

The process of shifting the tube position in forward projection and back projection is described as an example of the process of performing reconstruction while performing alignment has been described; however, examples are not limited thereto. For example, streak artifacts may be generated in an input image by shifting a projection image matrix or a projection path of the detector that is calculated by ray-tracing at the time of reconstruction.

The learning function 321 may acquire a target image by reconstructing the projection data H131 and acquire an input image by reconstructing the projection data H132 that is acquired from the same imaging subject as that of the projection data H131 while performing alignment on the projection data H132. The projection data H131 is an example of the first projection data. The projection data H132 is an example of the second projection data.

In the first to fourth embodiments described above, the case where streak artifacts are generated in the input image by performing alignment has been described. On the other hand, in a fifth embodiment, the case where streak artifacts are generated in an input image by reconstruction processing in which high-frequency components are enhanced will be described.

For example, the learning function 321 acquires a target image by reconstructing projection data H141 under a first reconstruction condition. The learning function 321 acquires an input image by reconstructing the projection data H141 under a second reconstruction condition. The second reconstruction condition is a reconstruction condition under which high-frequency components are enhanced more than under the first reconstruction condition. For example, under the first reconstruction condition, a normal reconstruction function represented by a Ramp function is used and, under the second condition, a reconstruction function that enhances high frequencies excessively compared to the Ramp function is used. Accordingly, the learning function 321 is able to generate streak artifacts in an input image that is reconstructed.

The learning function 321 may acquire a target image by reconstructing the projection data H141 under the first reconstruction condition and acquire an input image by reconstructing the projection data H142 that is acquired from the same imaging subject as that of the projection data H141 under the second reconstruction condition under which high frequencies are enhanced more than under the first reconstruction condition. The projection data H141 is an example of the first projection data. The projection data H142 is an example of the second projection data.

In the fifth embodiment, the case where streak artifacts are generated in the input image by the reconstruction processing in which high-frequency components are enhanced has been described. In other words, in the fifth embodiment, the case where streak artifacts are applied at the time of generation of an input image has been described. On the other hand, in a sixth embodiment, the case where streak artifacts are applied after image generation will be described.

For example, the learning function 321 acquires a target image by reconstructing projection data H151. The learning function 321 acquires an input image by performing the processing of enhancing high-frequency components on the target image. While the input images described in the first to fifth embodiment are generated by performing the artifact generation processing by the projection data domain, the input image in the sixth embodiment is generated by performing the artifact generation processing by an image data domain.

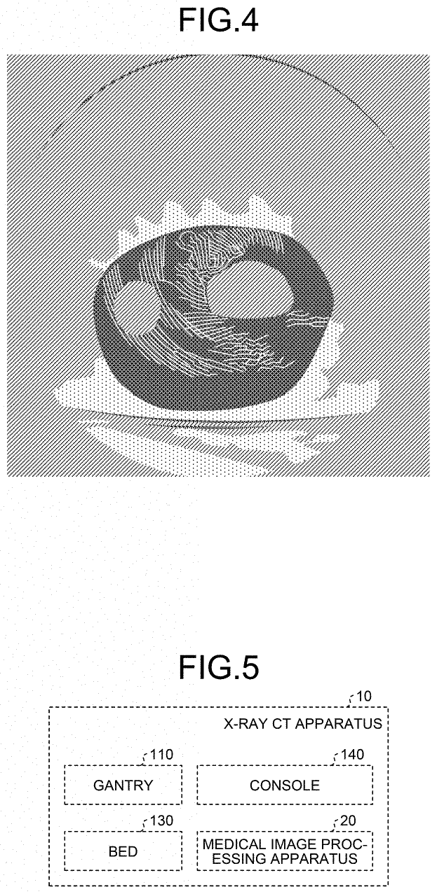

The input images illustrated in the first to fifth embodiments contain streak artifacts like those illustrated in FIG. 4. In other words, artifacts are generated such that streaks extend from the vicinity of a structure, such as a bone. On the other hand, the target images illustrated in the first to fifth embodiments are approximately the same image as that of FIG. 4 except that streak artifacts are not contained. Accordingly, subtraction between the target images and the input images in the first to fifth embodiments makes it possible to generate images presenting streak artifacts. It is also possible to specify a frequency corresponding to the streak artifacts based on the image presenting the streak artifacts. By amplifying frequency components corresponding to streak artifacts in the target image obtained by reconstructing the projection data H151, the learning function 321 is able to acquire an input image containing streak artifacts.

The learning function 321 may acquire a target image by reconstructing the projection data H151 under the first reconstruction condition and acquire an input image containing streak artifacts by performing the processing of enhancing high-frequency components on a reconstruction image obtained by reconstructing the projection data H152 that is acquired from the same imaging subject as that of the projection data H151. The projection data H151 is an example of the first projection data. The projection data H152 is an example of the second projection data.

Various modes may be carried out in addition to the above-described embodiments.

For example, FIGS. 1A and 1B illustrate that the processing circuitry 32 of the medical image processing apparatus 30 executes the learning function 321 and the processing circuitry 24 of the medical image processing apparatus 20 executes the image processing function 242. In other words, FIGS. 1A and 1B illustrate the case where the processing of generating the model M1 and the processing of reducing artifacts are executed in different apparatuses.

The processing of generating the model M1 and the processing of reducing artifacts may be executed in the same apparatus. For example, the processing circuitry 24 of the medical image processing apparatus the processing of generating the model M1 performed by the learning function 321 and the processing of reducing artifacts performed by the image processing function 242 may be executed in the medical image processing apparatus 20.

FIG. 1A, illustrates the X-ray CT apparatus 10 and the medical image processing apparatus 20 as independent apparatuses; however, as illustrated in FIG. 5, the medical image processing apparatus 20 may be contained in the X-ray CT apparatus 10. FIG. 5 is a block diagram illustrating an example of the configuration of the X-ray CT apparatus 10 according to a seventh embodiment.

In FIG. 5, the console 140 and the medical image processing apparatus 20 may be partly or entirely integrated. For example, any one of the display 142 of the console 140 and the display 22 of the medical image processing apparatus 20 may be omitted. Any one of the processing circuitry 144 of the console 140 and the processing circuitry 24 of the medical image processing apparatus 20 may be omitted. For example, the processing circuitry 24 may be omitted and the processing circuitry 144 may execute the acquiring function 241, the image processing function 242, and the outputting function 243 described above. Furthermore, the processing circuitry 144 may execute the learning function 321 described above.

FIG. 1A illustrates the X-ray CT image as an example of the radiographic image, and various radiographic images, such as a PET image or a SPECT image, apply similarly. Even in the case not associated with the reconstruction processing, such as the case were a two-dimensional X-ray image is acquired by a chest X-ray or X-ray angiography apparatus, the above-described embodiment is partly applicable. For example, it is possible to generate the model M1 by performing machine learning using an X-ray image that is acquired by chest X-ray as a target image and using an image obtained by performing the processing of enhancing high-frequency components on the target image as an input image.

The word “processor” used in the description given above refers to, for example, a circuit, such as a CPU, a GPU (Graphics Processing Unit), an application specific integrated circuit (ASIC), or a programmable logic device (for example, a simple programmable logic device (SPLD), a complex programmable logic device (CPLD) or a field programmable gate array (FPGA)). When the processor is, for example, a CPU, the processor reads programs that are saved in a storage circuit and executes the programs, thereby implementing the functions. On the other hand, when the processor is, for example, an ASIC, instead of saving the programs in the storage circuit, the functions are directly installed as a logic circuit in the circuit of the processor. Each processor of the embodiments is not limited to the case where each processor is configured as a single circuit, and multiple independent circuits may be combined to configure a single processor to implement the functions. Furthermore, the components in each drawing may be integrated into one processor to implement functions thereof.

FIG. 1A illustrates that the single memory 21 stores the programs corresponding to the respective processing functions of the processing circuitry 24. FIG. 1B illustrates that the single memory 31 stores the programs corresponding to the respective processing functions of the processing circuitry 32. FIG. 2 illustrates that the single memory 141 stores the programs corresponding to the respective processing functions of the processing circuitry 144. Embodiments however are not limited to this. For example, a plurality of the memories 21 may be arranged in a distributed manner and the processing circuitry 24 may be configured to read a corresponding program from the particular memory 21. Similarly, a plurality of the memories 31 may be arranged in a distributed manner and the processing circuitry 32 may be configured to read a corresponding program from the particular memory 31. Similarly, a plurality of the memories 141 may be arranged in a distributed manner and the processing circuitry 144 may be configured to read a corresponding program from the particular memory 141. Instead of saving the programs in the memory 21, the memory 31 or the memory 141, the programs may be directly installed in a circuit of a processor. In this case, the processor reads the programs that are installed in the circuit and executes the programs, thereby implementing the functions.

Each of the components of each device according to the above-described embodiments is a functional idea and thus need not necessarily be configured physically as unillustrated in the drawings. In other words, specific modes of distribution and integration of the devices are not limited to those illustrated in the drawings, and all or part of the devices may be configured in a distributed or integrated manner functionally or physically in any unit according to various types of load and the usage. Furthermore, all or any part of the processing functions implemented by the respective devices may be implemented by a CPU and programs that are analyzed and executed by the CPU or may be implemented as hardware using a wired logic.

It is possible to implement the medical image processing method described in the above-described embodiments by executing a program that is prepared in advance with a computer, such as a personal computer or a work station. The program can be distributed via a network, such as the Internet. The program may be recorded in a computer-readable and non-transient recording medium, such as a hard disk, a flexible disk (FD), a CD-ROM, a MO or a DVD, and may be read from the recording medium by the computer and thus be executed.

According to at least one of the embodiments described above, it is possible to reduce streak artifacts in a radiographic image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
processing circuitry configured to
acquire a radiographic image of a subject and
acquire a post-processing image with reduced steak artifacts by applying a model that reduces streak artifacts to the radiographic image,
wherein the model is generated by machine learning that uses, as a training data pair, a first radiographic image and a second radiographic image based on artifact generation processing for generating streak artifacts.

2. The medical image processing apparatus according to claim 1, wherein the processing circuitry is configured to further generate the model.

3. The medical image processing apparatus according to claim 2, wherein the processing circuitry is configured to perform the artifact generation processing by a projection data domain.

4. The medical image processing apparatus according to claim 2, wherein the processing circuitry is configured to acquire the second radiographic image by performing alignment as the artifact generation processing.

5. The medical image processing apparatus according to claim 4, wherein the processing circuitry is configured to acquire the first radiographic image by reconstructing projection data and acquire the second radiographic image by performing alignment on the projection data and reconstructing the projection data after the alignment.

6. The medical image processing apparatus according to claim 4, wherein the processing circuitry is configured to acquire the first radiographic image by reconstructing first projection data and acquire the second radiographic image by performing alignment on second projection data that is acquired from the same imaging subject as that of the first projection data and reconstructing the projection data after the alignment.

7. The medical image processing apparatus according to claim 4, wherein the processing circuitry is configured to acquire the first radiographic image by reconstructing first projection data and acquire the second radiographic image by reconstructing second projection data that is acquired while performing alignment on the same imaging subject as that of the first projection data.

8. The medical image processing apparatus according to claim 4, wherein the processing circuitry is configured to acquire the first radiographic image by reconstructing first projection data and acquire the second radiographic image by reconstructing the first projection data or second projection data that is acquired from the same imaging subject as that of the first projection data while performing alignment.

9. The medical image processing apparatus according to claim 2, wherein the processing circuitry is configured to acquire the first radiographic image by reconstructing first projection data under a first reconstruction condition and acquire the second radiographic image by reconstructing the first projection data or second projection data that is acquired from the same projection subject as that of the first projection data under a second reconstruction condition that high frequency components are enhanced more than under the first reconstruction condition.

10. The medical image processing apparatus according to claim 2, wherein the processing circuitry is configured to perform the artifact generation processing by an image data domain.

11. The medical image processing apparatus according to claim 2, wherein the processing circuitry is configured to acquire the first radiographic image by reconstructing first projection data under a first reconstruction condition and acquire the second radiographic image by performing processing of enhancing high-frequency components on the first radiographic image or a reconstruction image obtained by reconstructing second projection data that is acquired from the same imaging subject as that of the first projection data.

12. A medical image processing apparatus comprising processing circuitry configured to generate a model that reduces streak artifacts by executing machine learning that uses, as a training data pair, a first radiographic image and a second radiographic image based on artifact generation processing for generating streak artifacts.

13. An X-ray CT apparatus comprising the medical image processing apparatus according to claim 1.

14. A medical image processing method comprising:
acquiring a radiographic image of a subject; and
acquiring a post-processing image with reduced steak artifacts by applying a model that reduces streak artifacts to the radiographic image,
wherein the model is generated by machine learning that uses, as a training data pair, a first radiographic image and a second radiographic image based on artifact generation processing for generating streak artifacts.

15. A non-volatile storage medium storing a program that cause a computer to execute the medical image processing method according to claim 14.

16. The medical image processing apparatus according to claim 1, wherein the streak artifacts are aliasing or artifacts resulting from a positional shift of a radiographic imaging system that is used to acquire the radiographic image.

17. The medical image processing apparatus according to claim 12, wherein the streak artifacts are artifacts resulting from aliasing or a positional shift of a radiographic imaging system that is used to acquire the radiographic image.

18. The medical image processing method according to claim 14, wherein the streak artifacts are artifacts resulting from aliasing or a positional shift of a radiographic imaging system that is used to acquire the radiographic image.

19. A medical image processing system comprising:
the medical image processing apparatus according to claim 1; and
a radiography apparatus configured to perform scanning on a subject to generate a radiographic image to be processed by the medical image processing apparatus.

20. The medical image processing system according to claim 19, wherein the processing circuitry is configured to reconstruct the radiographic image based on subject data that is acquired by the scanning.
